(19) United States
(12) Patent Application Publication
(10) Pub. No.: US 2021/0155972 A1
(43) Pub. Date: May 27, 2021

(54) TARGETED RARE ALLELE CRISPR ENRICHMENT

(71) Applicant: GENETICS RESEARCH, LLC, Waltham, MA (US)

(72) Inventor: Anthony P. Shuber, Northbridge, MA (US)

(21) Appl. No.: 17/102,855

(22) Filed: Nov. 24, 2020

Related U.S. Application Data
(60) Provisional application No. 62/941,181, filed on Nov. 27, 2019.

(51) Int. Cl. C12Q 1/683; C12Q 1/6886

(52) U.S. Cl. CPC C12Q 1/683; C12Q 1/6886; C12Q 2600/118; C12Q 2600/106; C12Q 2600/156

(57) ABSTRACT

Methods of detecting a mutation comprise introducing a Cas endonuclease complex to a nucleic acid sample, wherein guide RNA in the Cas endonuclease complex bind to a location of a suspected mutation. Unbound nucleic acid in the sample is degraded or separated from the bound complex, and presence of the mutation is detected by detecting bound Cas endonuclease complex. The Cas endonuclease complex comprises a Cas endonuclease and guide RNA. The guide RNA is designed to bind to the location of the suspected mutation. In some instances, the Cas endonuclease complex comprises a detectable label, such as a fluorescent label. Therefore, detecting presence of the mutation comprises detecting presence of the label. An exonuclease may be used to degrade or digest unbound nucleic acid and isolate the mutation. Methods include further analysis of the isolated mutation.

### CROSS-REFERENCE TO RELATED APPLICATIONS

This application claims the benefit of U.S. Provisional Application 62/941,181, filed Nov. 27, 2019, the contents of which are hereby incorporated by reference in their entirety.

### TECHNICAL FIELD

The invention relates to molecular genetics and detection of nucleic acids.

### BACKGROUND

Laboratories are increasingly using DNA and RNA for clinical analysis. For example, DNA can reveal whether a person has a disease-associated mutation, or is a carrier of a hereditable disease. Fetal DNA can be studied to detect inherited genetic disorders and aneuploidy. However, a consistent challenge in accessing actionable genomic information lies in existing approaches to detecting very rare mutations, i.e., mutant alleles of DNA present only in very small frequencies among large populations of DNA.

Methods of detecting mutations often include tests based on DNA sequencing and the use of next-generation sequencing (NGS) platforms to capture, amplify, and sequence a subject's DNA. However, typical NGS platforms face a number of challenges. Detecting rare mutations in samples that also contain an abundance of wild-type DNA requires successfully amplifying rare DNA species. Given the stochastic nature of PCR, the ability to amplify rare fragments has been a challenge. Other detection methods, such as using fluorescent probe hybridization, face similar challenges. For example, when a mutation is present in quantities as low as hundredths of a percent of copies present, probe assays may miss the mutation entirely.

### SUMMARY

Methods of the invention allow for detection of rare mutations or mutations present at a low frequency (0.1%). Methods of the invention use a CRISPR-based enrichment to target and detect a mutation in a sample from a patient. For the CRISPR enrichment, a Cas complex or ribonucleic protein (RNP) complex is added to the sample. Guide RNA (gRNA) in the Cas complex or RNP complex is designed to bind with the suspected mutation. If the target is present, a CRISPR-associated (Cas) endonuclease in the Cas complex protects the target while any unprotected nucleic acid in the sample is removed (e.g., by a wash or separation) or degraded (e.g., by an exonuclease). After removal/degradation, what remains of the nucleic acid sample is then detected by any suitable means.

An important benefit of targeted rare allele CRISPR enrichment according to the disclosure is the ability to successfully detect a very rare allele in the presence of an arbitrarily large amount of a dominant allele. For example, a sample may include many copies of a gene of interest in which about, to illustrate, 99.9% are wild type, while 0.1% differ by a single base and thus represent a rare allele. The disclosure includes the insight that a ribonucleoprotein (RNP) comprising a Cas endonuclease and a guide RNA specific to the single base may be successfully used to “capture” and detect the rare allele. Methods of the disclosure are useful for detecting rare alleles and applicable to sample types such as cell-free nucleic acid in blood or plasma. It may be understood that blood or plasma from a patient with cancer may contain circulating tumor DNA (ctDNA), which may represent a very rare minor allele in the presence of abundant, homologous wild type DNA. Using guide RNA designed to hybridize specifically to a mutation in the ctDNA, one may detect and even quantify ctDNA using an RNP of the disclosure.

Methods of the invention are useful in a wide variety of applications. For example, because methods of the invention preserve target sequence, they are ideal for detection of sequence that is present in a sample at low abundance. Thus, methods of the invention are useful for analysis of cfDNA in blood or blood products (e.g., plasma). As a result, methods of the invention allow the early detection of genomic alterations indicative of cancer and identification of genetic disorders of a fetus in utero. In some examples, the mutation is a base-pair substitution, deletion or insertion of a single base pair, or single nucleotide polymorphism (SNP).

In some embodiments, methods of the invention allow for rapid detection of a mutation. In some embodiments, the Cas complex comprises a detectable label, and presence of the detectable label indicates presence of the mutation. For example, the detectable label may be a fluorescent label. Detection of the fluorescent label indicates presence of the mutation in the sample, and no further sequencing or PCR steps are needed for such detection. In some embodiments, microscopy may be used for detection of the detectable label after degradation of the unprotected nucleic acid.

In an embodiment, the invention provides methods of detecting a target nucleic acid by enrichment. In an embodiment, the target nucleic acid is a mutation, such as a single base mismatch. The methods include protecting a target nucleic acid in a sample and optionally removing or degrading unprotected nucleic acids. Protection can be mediated by Cas endonuclease complexes. Preferably, the target nucleic acid is detected in a sample. The sample may include an abundance of a predominant or “wild type” allele. The method is useful where the target nucleic acid represents less than about 1% of the copies of the gene present in the sample, while the predominant allele represents at least about 99% of the copies. Methods of the disclosure may be used where the target nucleic acid represents less than about 0.1% of the copies of the gene present in the sample, while the predominant allele represents at least about 99.9% of the copies. Where a patient with cancer has previously had tumor DNA sequenced, and the patient has undergone a treatment for the cancer, methods of the disclosure may be employed to detect a tumor-specific allele of cell-free ctDNA in a blood or plasma sample from the patent. The method may include designing guide RNA specific to the tumor-specific allele, and introducing RNP (comprising a Cas endonuclease and the guide RNA) into a blood or plasma sample from the patient. The RNP binds to the tumor-specific allele. The RNP-bound tumor-specific allele may then be detected to indicate the presence of the tumor-specific allele in the blood or plasma sample from the patient. For example, the sample may be subject to a size separation (e.g., on a gel or column) to remove unbound nucleic acid, or the sample may be enriched for the rare target by digesting unbound nucleic acid with exonuclease while the RNP binds to, and protects, the rare allele. The methods may include detecting the protected nucleic acids. The Cas endonuclease complex attaches to a target nucleic acid to protect the target in the nucleic acid sample.

The method further comprises degrading unprotected nucleic acid in the sample. In an embodiment, an exonuclease is introduced to the sample to digest the unbound nucleic acid in the sample. Preferably, all of the unprotected nucleic acids are degraded. Preferably, the protected nucleic acids include the target nucleic acid. For example, one or more exonucleases may be introduced that promiscuously digest unbound, unprotected nucleic acid. While the exonucleases act, the segment containing the target nucleic acid of interest is protected by the bound complexes and survives the digestion step intact. The exonuclease may be deactivated after a prescribed time period that allows for the unprotected nucleic acid to be digested or degraded. If nucleic acid remains after the digestion or degradation, the nucleic acid is the target. Thus, methods of the invention provide for isolated target nucleic acid. The isolated target can be removed from Cas by known laboratory techniques, including heating, chemical denaturation, sonic, or any suitable method, including wash steps.

Degradation of unprotected nucleic acids may include digestion with an exonuclease, such as exonuclease I, exonuclease II, exonuclease III, exonuclease IV, exonuclease V, exonuclease VI, exonuclease VII, or exonuclease VIII. In certain embodiments of the invention, the exonuclease is deactivated after a portion of the nucleic acid is digested. If left to completion, the exonuclease would digest all, or nearly all, of the unprotected nucleic acid. In some instances, heat is used to deactivate the exonuclease so that the exonuclease stops digesting non-target nucleic acid in the sample.

Methods of the invention further include detecting the target sequence. In some embodiments, the method comprises detecting presence of the mutation by detecting bound Cas endonuclease complex. In some embodiments, binding proteins (Cas proteins) may be removed prior to detection. The undamaged portion (i.e., that portion that was protected or otherwise not degraded by exonuclease digestion) may be detected by any means known in the art. For example and without limitation, the intact portion may be detected by DNA staining, spectrophotometry, sequencing, fluorescent probe hybridization, fluorescence resonance energy transfer, optical microscopy, or electron microscopy.

In some embodiments, the method further comprises a wash step to isolate the mutation. In some embodiments, the method comprises further analysis of the isolated mutation. Further analysis comprises any of hybridization, spectrophotometry, sequencing, electrophoresis, amplification, fluorescence detection, and chromatography.

CRISPR-associated (Cas) complexes are used in methods of the invention. The Cas complexes comprise guide RNA and a Cas endonuclease. Cas is complexed with target nucleic acid using guide RNAs that are designed for sequence-specific binding. An ideal protein is catalytically-inactive (dead) Cas (dCas). The method comprises introducing a Cas endonuclease complex to a nucleic acid sample. The guide RNA (gRNA) in the Cas endonuclease complex binds to a location of a suspected mutation. The Cas endonuclease complex comprises a Cas endonuclease and guide RNA. The guide RNA is designed to bind to the location of the suspected mutation.

In some embodiments of the invention, the proteins that bind to the target nucleic acid may be a Cas endonuclease or any proteins that bind a nucleic acid in a sequence-specific manner and protect sequence from degradation. The Cas endonuclease is complexed with target nucleic acid using guide RNAs that are designed for sequence-specific binding. An ideal protein is catalytically-inactive (dead) Cas (dCas). Preferably, the Cas complexes are Cas9 complexes. The Cas complexes include a Cas endonuclease and a guide RNA. The Cas endonuclease may include any Cas endonuclease. For example, the Cas endonuclease may be Cas9, Cas13, Cpf1, C2c1, C2c3, C2c2, CasX, or CasY, including modified versions of Cas9, Cas13, Cpf1, C2c1, C2c3, C2c2, CasX, or CasY in which the amino acid sequence has been altered. The Cas endonuclease is catalytically inactive. For example, the Cas endonuclease may be Streptococcus pyogenes Cas9 that has a D10A and/or a R1335K mutation, Acidaminococcus sp. BV3L6 Cpf1 that has a D908 mutation, or Lachnospiraceae bacterium ND2006 that has a D832 mutation. In some embodiments, the Cas endonuclease comprises a Cas9 protein. In some embodiments, the Cas9 protein comprises a catalytically inactive Cas9 protein.

The guide RNAs may be any guide RNA that functions with a Cas endonuclease. Individual guide RNAs may include a separate crRNA molecule and tracrRNA molecule, or individual guide RNAs may be single molecules that include both crRNA and tracrRNA sequences.

Any suitable sample may be analyzed using methods of the invention. In some embodiments, the sample is a human sample. Nucleic acid for analysis may be obtained from any sample type, such as a liquid or body fluid from a subject. In some embodiments, the sample is urine, blood, plasma, serum, sweat, saliva, semen, feces, phlegm, or a liquid biopsy. The nucleic acid may contain a mutation. For example and without limitation, the mutation may be a base-pair substitution, deletion or insertion of a single base pair, or single nucleotide polymorphism (SNP). The nucleic acid of interest may be from an infectious agent or pathogen. For example, the nucleic acid sample may be obtained from an organism, and the nucleic acid of interest may contain a sequence foreign to the genome of that organism. The nucleic acid of interest may be from a sub-population of nucleic acid within the nucleic acid sample. For example, the nucleic acid of interest may be cell-free DNA, such as cell-free fetal DNA or circulating tumor DNA. The nucleic acid may be any naturally-occurring or artificial nucleic acid. The nucleic acid may be DNA, RNA, hybrid DNA/RNA, peptide nucleic acid (PNA), morpholino and locked nucleic acid (LNA), glycol nucleic acid (GNA), threose nucleic acid (TNA), or Xeno nucleic acid. The RNA may be a subpopulation of RNA, such as mRNA, tRNA, rRNA, miRNA, or siRNA. Preferably the nucleic acid is DNA.

The sample may come from any source. For example, the source may be an organism, such as a human, non-human animal, plant, or other type of organism. The source may be a tissue sample from an animal, such as blood, serum, plasma, skin, conjunctiva, gastrointestinal tract, respiratory tract, vagina, placenta, uterus, oral cavity or nasal cavity. The source may be an environmental source, such as a soil sample or water sample, or a food source, such as a food sample or beverage sample. The sample may comprise nucleic acids that have been isolated, purified, or partially purified from a source. Alternatively, the sample may not have been processed.

The invention provides methods of detecting nucleic acid in a heterogenous population of nucleic acids by degrading non-target nucleic acids, making detection of the target more likely. Detection involves a form of negative enrichment in which target nucleic acid is protected and a selective enzymatic digestion of unprotected DNA or RNA is performed. In a preferred embodiment, target DNA or RNA is protected using Cas/Ribonucleic protein (RNP) complexes. Then, when the sample is exposed to a degradative enzyme, for example an exonuclease, unprotected ends are digested. Because the nucleic acid of interest has been isolated, simply detecting the presence of the target nucleic acid confirms the presence of the target or mutation. In some examples, the target or mutation is a single base mismatch in a subject or sample. Thus, the invention provides methods for rapidly and simply detecting a mutation in a complex sample, regardless of the presence of nucleic acids from other sources.

### BRIEF DESCRIPTION OF THE DRAWINGS

FIG. 1 shows an exemplary method of the invention.

FIG. 2 depicts a portion of nucleic acid during an exemplary method of the invention.

FIG. 3 depicts a portion of nucleic acid during an exemplary method of the invention.

FIG. 4 shows an embodiment of a kit according to the invention.

FIG. 5 shows results from an exemplary method of the invention.

### DETAILED DESCRIPTION

Methods of detecting a mutation comprise introducing a Cas endonuclease complex to a nucleic acid sample, wherein guide RNA in the Cas endonuclease complex bind to a location of a suspected mutation. Unbound nucleic acid in the sample is degraded, and presence of the mutation is detected by detecting bound Cas endonuclease complex. The Cas endonuclease complex comprises a Cas endonuclease and guide RNA. The guide RNA is designed to bind to the location of the suspected mutation. In some instances, the Cas endonuclease complex comprises a detectable label, such as a fluorescent label. Therefore, detecting presence of the mutation comprises detecting presence of the label. An exonuclease may be used to degrade or digest unbound nucleic acid and isolate the mutation. Methods include further analysis of the isolated mutation.

Methods of the invention include an enrichment step, or negative enrichment step, that leaves the target intact and isolated as a segment of DNA. The methods are useful for the isolation of intact DNA fragments of any arbitrary length and may preferably be used in some embodiments to isolate (or enrich for) fragments of DNA. The DNA fragments may be analyzed by any suitable method such as simple detection (e.g., via staining with ethidium bromide) or by single-molecule sequencing. Embodiments of the invention provide kits that may be used in performing methods described herein.

Methods of the invention are useful in a wide variety of applications. For example, because methods of the invention preserve target sequence, they are ideal for detection of sequence that is present in a sample at low abundance. Thus, methods of the invention are useful for analysis of cfDNA in blood or blood products (e.g., plasma). As a result, methods of the invention allow the early detection of genomic alterations indicative of cancer and identification of genetic disorders of a fetus in utero.

In some embodiments of methods of the invention, the target nucleic acid may be detected by first using Cas endonuclease to degrade substantially all nucleic acid in a sample except for the nucleic acid of interest, then detect the presence of the nucleic acid of interest. In some embodiments of methods of the invention, Cas endonuclease complexes are used to protect the nucleic acid of interest while unprotected nucleic acid is digested, e.g., by exonuclease, followed by detecting the nucleic acid of interest that remains. The invention provides methods of detecting a nucleic acid of interest in a population of nucleic acids by eliminating all of the nucleic acids other than the one of interest. Because the methods of the invention do not require “fishing” target nucleic acids from a population, they avoid problems of target size, sensitivity, and target adulteration associated with methods that rely on hybrid capture or PCR amplification.

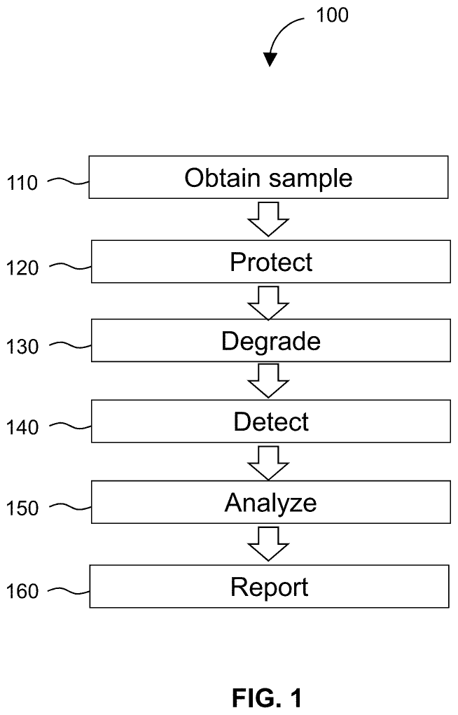

FIG. 1 shows an exemplary method of the invention. The method 100 comprises protecting 120 target nucleic acid in the sample. In some embodiments of the invention, binding proteins are used to protect a target nucleic acid in, or prepared from, a biological sample. Methods include binding proteins to a location of a suspected mutation or target in the nucleic acid sample. The proteins that bind to the target nucleic acid may be any proteins that bind a nucleic acid in a sequence-specific manner. In some embodiments, any suitable endonuclease is used. In some embodiments, the nuclease is a programmable nuclease.

Preferably, the endonuclease is a CRISPR-associated (Cas) endonuclease. Any suitable Cas endonuclease or homolog thereof may be used. For example, the Cas endonuclease may be Cas9, Cas13, Cpf1, C2c1, C2c3, C2c2, CasX, or CasY, including modified versions of Cas9, Cas13, Cpf1, C2c1, C2c3, C2c2, CasX, or CasY in which the amino acid sequence has been altered. The binding protein may be a catalytically inactive form of a nuclease, such as a programmable nuclease described above. For example and without limitation, the Cas endonuclease may be Streptococcus pyogenes Cas9 that has a D10A and/or R1335K mutation, Acidaminococcus sp. BV3L6 Cpf1 that has a D908 mutation, or Lachnospiraceae bacterium ND2006 that has a D832 mutation.

The binding protein may be complexed with a nucleic acid that guides the protein to a location of the nucleic acid suspected of having a mutation. For example, the protein may be a Cas endonuclease in a complex with a guide RNA. A guide RNA mediates binding of the Cas complex to the guide RNA target site via a sequence complementary to a sequence in the target site. Typically, guide RNAs that exist as single RNA species comprise a CRISPR (cr) domain that is complementary to a target nucleic acid and a tracr domain that binds a CRISPR/Cas protein. However, guide RNAs may contain these domains on separate RNA molecules. The guide RNAs may be any guide RNA that functions with a Cas endonuclease. Individual guide RNAs may include a separate crRNA molecule and tracrRNA molecule, or individual guide RNAs may be single molecules that include both crRNA and tracrRNA sequences.

Programmable nucleases and their uses are described in, for example, Zhang F, Wen Y, Guo X (2014). “CRISPR/Cas9 for genome editing: progress, implications and challenges”. Human Molecular Genetics. 23 (R1): R40-6. doi:10.1093/hmg/ddu125; Ledford H (March 2016). “CRISPR: gene editing is just the beginning”. Nature. 531 (7593):156-9. doi:10.1038/531156a; Hsu P D, Lander E S, Zhang F (June 2014). and “Development and applications of CRISPR-Cas9 for genome engineering”. Cell. 157 (6): 1262-78. doi:10.1016/j.cell.2014.05.010; Boch J (February 2011), the contents of each of which are incorporated herein by reference.

The method 100 further comprises digesting or degrading 130 unprotected nucleic acid in the sample by introducing an exonuclease. The exonuclease is deactivated after a portion of the unprotected nucleic acid in the sample is degraded or digested. Binding of the Cas complexes to the target provides protection against exonuclease digestion. Nucleic acids in the sample population are then degraded, but the target is protected from degradation. Preferably, degradation occurs via exonuclease digestion. Degradation of unprotected nucleic acids may occur by any suitable means. Preferably, unprotected nucleic acids are degraded by digestion with an exonuclease, such as exonuclease I, exonuclease II, exonuclease III, exonuclease IV, exonuclease V, exonuclease VI, exonuclease VII, or exonuclease VIII. Digestion may destroy a portion of the nucleic acids in the population other than the target. For example, digestion may degrade nucleic acids to individual nucleotides or to small fragments that are distinguishable from the intact target. After a period of time sufficient to degrade at least a portion of the nucleic acid that is not the target of interest, the exonuclease is deactivated. The exonuclease may be deactivated by any suitable means. For example, heat may be used to deactivate the exonuclease.

The method 100 further comprises detecting 140 the target. The target may be detected by any means known in the art. For example and without limitation, the target may be detected by DNA staining, spectrophotometry, sequencing, fluorescent probe hybridization, fluorescence resonance energy transfer, optical microscopy, or electron microscopy. Methods of DNA sequencing are known in the art and described in, for example, Pettersson E, Lundeberg J, Ahmadian A (February 2009). “Generations of sequencing technologies”. Genomics. 93 (2): 105-11. doi:10.1016/j.ygeno.2008.10.003; Goodwin, Sara; McPherson, John D.; McCombie, W. Richard (17 May 2016). “Coming of age: ten years of next-generation sequencing technologies”. Nature Reviews Genetics. 17 (6): 333-51. doi:10.1038/nrg.2016.49; and Morey M, Fernandez-Marmiesse A, Castiñeiras D, Fraga J M, Couce M L, Cocho J A (2013). “A glimpse into past, present, and future DNA sequencing”. Molecular Genetics and Metabolism. 110 (1-2): 3-24. doi:10.1016/j.ymgme.2013.04.024. Other methods of DNA detection are known in the art and described in, for example, Xu et al., Label-Free DNA Sequence Detection through FRET from a Fluorescent Polymer with Pyrene Excimer to SG, ACS Macro Lett., 2014, 3 (9), pp 845-848, DOI: 10.1021/mz500378c; and Green and Sambrook, eds., Molecular Cloning: A Laboratory Manual, 4th edition, Cold Spring Harbor Press, Cold Spring Harbor, N.Y., 2012, ISBN 978-1-936113-41-5.

The nucleic acid may be detected, sequenced, or counted. When multiple nucleic acids of interest are present, they may be quantified, e.g., by qPCR.

A feature of the method is that a specific target nucleic acid, such as a mutation, may be detected by a technique that includes detecting only the presence or absence of a fragment of DNA, and it need not be necessary to sequence DNA from a subject to describe mutations. The gRNA selects for a known mutation. If it doesn't find the mutation, no protection is provided and the molecule gets digested. The method is well suited for the analysis of small portions of DNA, degraded samples, samples in which the target of interest is extremely rare, and particularly for the analysis of maternal serum (e.g., for fetal DNA) or a liquid biopsy (e.g., for ctDNA).

In some embodiments, the invention provides methods of detecting nucleic acid in a heterogenous population of nucleic acids by degrading non-target nucleic acids, making detection of the target more likely. Detection involves a form of negative enrichment in which target nucleic acid is protected and a selective enzymatic digestion of unprotected DNA or RNA is performed. In a preferred embodiment, target DNA or RNA is protected using ribonucleoprotein (RNP) complexes that include Cas endonuclease and guide RNA. Then, when the sample is exposed to a degradative enzyme, for example an exonuclease, unprotected ends are digested. Because the target, or nucleic acid of interest, has been isolated, simply detecting the presence of the target confirms the presence of the mutation in a subject or sample. Thus, the invention provides methods for rapidly and simply detecting a mutation in a complex sample, regardless of the presence of nucleic acids from other sources.

In some embodiments, the nucleic acid may be provided as an aliquot (e.g., in a micro centrifuge tube such as that sold under the trademark EPPENDORF by Eppendorf North America (Hauppauge, N.Y.) or glass cuvette). The nucleic acid may be disposed on a substrate. For example, the nucleic acid may be pipetted onto a glass slide and subsequently combed or dried to extend it across the glass slide. The nucleic acid may optionally be amplified. Optionally, adaptors are ligated to ends of the nucleic acid, which adaptors may contain primer sites or sequencing adaptors. The presence of the nucleic acid may then be detected using an instrument. In certain embodiments, the instrument is a spectrophotometer, and detection includes measuring the adsorption of light by the nucleic acid. The method may be performed in fluid partitions, such as in droplets on a microfluidic device, such that each detection step is binary (or “digital”). For example, droplets may pass a light source and photodetector on a microfluidic chip and light may be used to detect the presence of a segment of DNA in each droplet (which segment may or may not be amplified as suited to the particular application circumstance). By the described methods, a sample can be assayed using a technique that is inexpensive, quick, and reliable. Methods of the disclosure are conducive to high throughput embodiments, and may be performed, for example, in droplets on a microfluidic device, to rapidly assay a large number of aliquots from a sample for one or any number of genomic structural alterations.

The method further comprises further isolation and analysis 150 of the target. The isolated target can be removed from Cas by known laboratory techniques, including heating, chemical denaturation, sonic, or any suitable method, including wash steps. Methods of the invention may include a wash step for purification at any time. For example, wash steps may include a wash on a column, a bead wash, and isolation or purification such as gel purification, e.g., by SDS-PAGE.

Once the isolated target is removed from Cas, the target may be further analyzed. In certain aspects of the invention, methods include further analysis of mutation. Methods of analysis of nucleic acids are known in the art and described, for example, in Green and Sambrook, Molecular Cloning: A Laboratory Manual (Fourth Edition), Cold Spring Harbor Laboratory Press, Woodbury, N.Y. 2,028 pages (2012), incorporated herein by reference. For example and without limitation, the target may be further analyzed using any of hybridization, spectrophotometry, sequencing, electrophoresis, amplification, fluorescence detection, or chromatography. Non-limiting examples of detection methods include PCR, hybrid capture, Next Generation Sequencing, and sequencing such as according to Pacific Biosciences, Oxford Nanopore, Helicos Biosciences, and optical sequencing.

Because methods of the invention work to capture very long (500, 1,000, 5,000 bases) targets, the methods are useful as sample preparation for sequencing technologies that can sequence very long nucleic acid fragments. For example, third generation sequencing technologies that offer long reads or can sequence long nucleic acid molecules. For example, Oxford Nanopore provides nanopore sequencing products for the direct, electronic analysis of single molecules.

In some embodiments, the method 100 comprises reporting 160 presence of the target. A report may be provided to a subject or patient. The report may provide results on presence or detection of the target. The report preferably includes information about the subject's condition, such as a diagnosis, prognosis, or suggested course of therapy.

In some embodiments, the method 100 comprises obtaining 110 the sample. Nucleic acid for analysis may be obtained from any sample type, such as a liquid or body fluid from a subject, such as urine, blood, plasma, serum, sweat, saliva, semen, feces, phlegm, or a liquid biopsy. The sample may be a food sample. The sample may be from an environmental source, such as a soil sample, or water sample. In some instances, the sample is a human sample. In some embodiments, the sample is a non-human animal sample.

The nucleic acid of interest may contain a mutation. For example and without limitation, the feature may be an insertion, deletion, substitution, inversion, amplification, duplication, translocation, or polymorphism. The nucleic acid of interest may be from an infectious agent or pathogen. For example, the nucleic acid sample may be obtained from an organism, and the nucleic acid of interest may contain a sequence foreign to the genome of that organism. The nucleic acid of interest may be from a sub-population of nucleic acid within the nucleic acid sample. For example, the nucleic acid of interest may be cell-free DNA, such as cell-free fetal DNA or circulating tumor DNA.

The population of nucleic acids may come from any source. The source may be an organism, such as a human, non-human animal, plant, or other type of organism. The source may be a tissue sample from an animal, such as blood, serum, plasma, skin, conjunctiva, gastrointestinal tract, respiratory tract, vagina, placenta, uterus, oral cavity or nasal cavity. The source may be an environmental source, such as a soil sample or water sample, or a food source, such as a food sample or beverage sample. Preferably, the target nucleic acid is detected in a sample. The sample may include an abundance of a predominant or “wild type” allele. The method is useful where the target nucleic acid represents less than about 1% of the copies of the gene present in the sample, while the predominant allele represents at least about 99% of the copies. Methods of the disclosure may be used where the target nucleic acid represents less than about 0.1% of the copies of the gene present in the sample, while the predominant allele represents at least about 99.9% of the copies. Where a patient with cancer has previously had tumor DNA sequenced, and the patient has undergone a treatment for the cancer, methods of the disclosure may be employed to detect a tumor-specific allele of cell-free ctDNA in a blood or plasma sample from the patent. The method may include designing guide RNA specific to the tumor-specific allele, and introducing RNP (comprising a Cas endonuclease and the guide RNA) into a blood or plasma sample from the patient. The RNP binds to the tumor-specific allele. The RNP-bound tumor-specific allele may then be detected to indicate the presence of the tumor-specific allele in the blood or plasma sample from the patient. For example, the sample may be subject to a size separation (e.g., on a gel or column) to remove unbound nucleic acid, or the sample may be enriched for the rare target by digesting unbound nucleic acid with exonuclease while the RNP binds to, and protects, the rare allele. The methods may include detecting the protected nucleic acids. The Cas endonuclease complex attaches to a target nucleic acid to protect the target in the nucleic acid sample.

The nucleic acid may be any naturally-occurring or artificial nucleic acid. The nucleic acid may be DNA, RNA, hybrid DNA/RNA, peptide nucleic acid (PNA), morpholino and locked nucleic acid (LNA), glycol nucleic acid (GNA), threose nucleic acid (TNA), or Xeno nucleic acid. The RNA may be a subpopulation of RNA, such as mRNA, tRNA, rRNA, miRNA, or siRNA. Preferably the nucleic acid is DNA.

The population of nucleic acids may have been isolated, purified, or partially purified from a source. Techniques for preparing nucleic acids from tissue samples and other sources are known in the art and described, for example, in Green and Sambrook, Molecular Cloning: A Laboratory Manual (Fourth Edition), Cold Spring Harbor Laboratory Press, Woodbury, N.Y. 2,028 pages (2012), incorporated herein by reference. Alternatively, the nucleic acids may be contained in sample that has not been processed. The nucleic acids may single-stranded or double-stranded. Double-stranded nucleic acids may be DNA, RNA, or DNA/RNA hybrids. Preferably, the nucleic acids are double-stranded DNA.

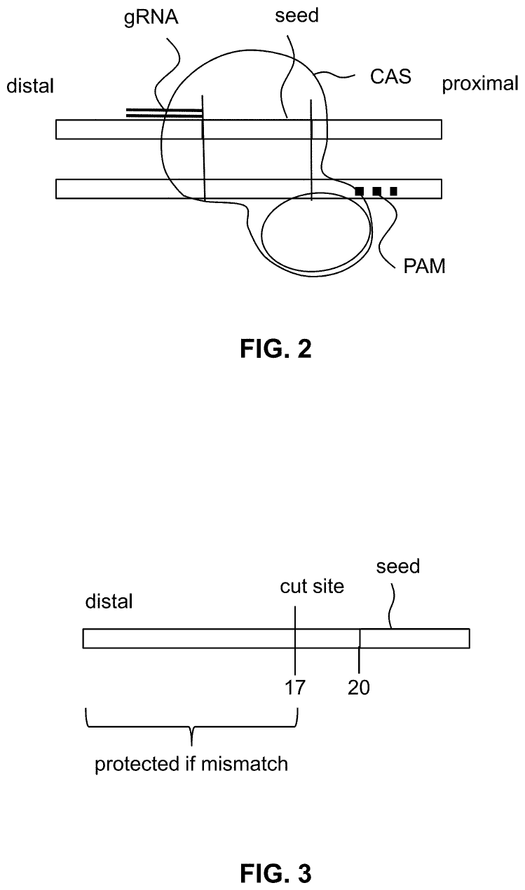

FIG. 2 depicts binding of a Cas9 complex to a target area of a nucleic acid. The nucleic acid has a distal end and a proximal end. The gRNA guides the Cas9 complex to the target location. The gRNA is specific to the mutation suspected. If the mutation is present, the gRNA will bind to the target nucleic acid. The seed is part of the nucleic acid sequence to which gRNA hybridizes. After the Cas9 complex binds to the mutation and protects the mutation, an exonuclease may be introduced to degrade or digest anything that is unprotected. The Cas9 complex may include a label. After exonuclease degradation or digestion, detection of the label on anything that is left in the sample indicates presence of the mutation.

The Cas9 recognizes the PAM, and the gRNA hybridizes to the seed region. The gRNA hybrid is not stable if there is a mismatch in the seed region. However, the Cas9 will still bind and cut—the Cas endonuclease will protect the gRNA side, and the gRNA will release due to instability from the mismatch in the seed region. If there is a perfect match in the seed region, then the Cas endonuclease will cut and protect both ends. The cut site or cleavage site is 3 base pair upstream from the PAM sequence.

As shown in FIG. 3, if the suspected mutation is present, the distal end is protected by the Cas endonuclease after cutting or cleavage of the Cas complex. The cut site is at bp 17, three base pair upstream from the seed.

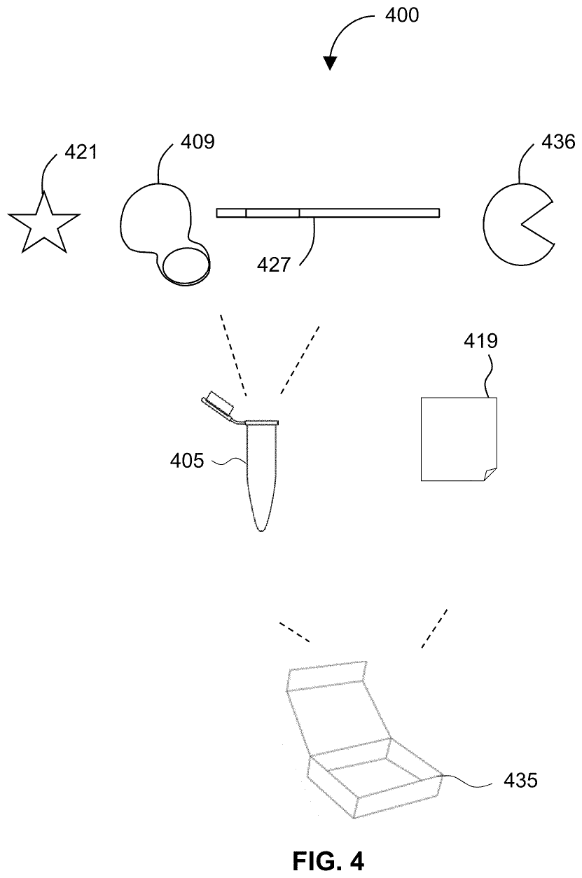

FIG. 4 shows a kit 400 of the invention. The kit 400 may include reagents 405 for performing the steps described herein. For example, the reagents 405 may include one or more of a Cas endonuclease 410, a guide RNA 415, a detectable label 420, and exonuclease 425. The kit 400 may also include instructions 430 or other materials. The reagents 405, instructions 430, and any other useful materials may be packaged in a suitable container 435. Kits of the invention may be made to order. For example, an investigator may use, e.g., an online tool to design guide RNA and reagents for the performance of methods of the invention. The guide RNAs 415 may be synthesized using a suitable synthesis instrument. The synthesis instrument may be used to synthesize oligonucleotides such as gRNAs or single-guide RNAs (sgRNAs). Any suitable instrument or chemistry may be used to synthesize a gRNA. In some embodiments, the synthesis instrument is the MerMade 4 DNA/RNA synthesizer from Bioautomation (Irving, Tex.). Such an instrument can synthesize up to 12 different oligonucleotides simultaneously using 50, 200, or 1,000 nanomole prepacked columns. The synthesis instrument can prepare a large number of guide RNAs 415 per run. These molecules (e.g., oligos) can be made using individual prepacked columns (e.g., arrayed in groups of 96) or well-plates. The resultant reagents 405 (e.g., guide RNAs 415, endonuclease(s) 410, detectable label(s) 420, and exonucleases 425) can be packaged in a container 435 for shipping as a kit.

### Example

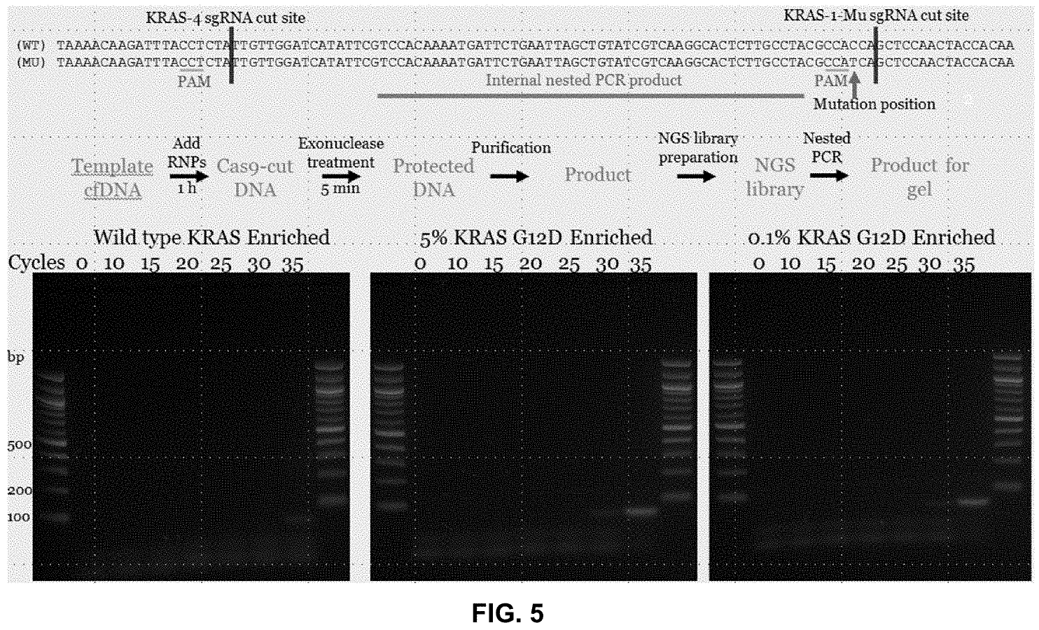

FIG. 5 shows an exemplary method of the invention for detecting a KRAS mutation. The wild-type KRAS is the natural, unchanged form of the gene that makes a protein called KRAS and is involved in cell signaling pathways that control cell growth, cell maturation, and cell death. Mutated forms of the KRAS gene have been found in some types of cancer.

Allele specific methods of the invention were used to detect 0.1% mutant variant by gel electrophoresis. The method comprises starting with a template cell free DNA (cfDNA) sample, adding ribonucleic proteins (RNP) for about 1 hour to allow for Cas9 to cut the DNA. The method continues with exonuclease treatment for about 5 minutes to achieve protected DNA. The method then goes through purification to arrive at a product, which may then go through Next Generation Sequencing (NGS) library preparation to prepare a NGS library which goes through nested polymerase chain reaction (PCR) to provide a product for gel.

The (WT) wild type KRAS DNA is shown on the top row, with the (MU) mutation KRAS DNA shown immediately below. The sgRNA cut sites are shown, as well as the mutation position and the PAM. FIG. 5 also shows three gels. Each gel has 9 lanes, with the first lane and the ninth lane showing the ladders. The second lane shows 0 PCR cycles, the third lane shows 10 PCR cycles, the fourth lane shows 15 PCR cycles, the fifth lane shows 20 PCR cycles, the sixth lane should 25 PCR cycles, the seventh lane shows 30 PCR cycles, and the eighth lane shows 35 PCR cycles. The gel titled Wild type KRAS Enriched shows a faint band in the eighth lane for a product. The gel titled 5% KRAS G12D Enriched shows a bright band in the eighth lane for a product (here, a mutation), indicating that the product is present and has been amplified through 35 PCT cycles. The gel titled 0.1% KRAS G12D Enriched also shows a bright band in the eighth lane, indicating that the product is present and has been amplified through 35 PCT cycles, even when the product is rare or present at a low frequency (0.1%).

In another example, methods of the disclosure involve assaying a sample for tumor DNA. A patient suspected of having cancer may have DNA sequenced to identify tumor DNA and, in some cases, the sequencing may identify “matched normal” DNA. That is to say, during a course of working with a patient with cancer, clinicians may identify tumor-specific mutations or alleles and may also identify the corresponding wild-type allele present in healthy, non-cancer cells of the patient. The tumor allele may be understood to be a marker of the presence of cancer in the patient. In particular, it may be understood that tumor DNA circulates in the patient's bloodstream as cell-free circulating DNA fragments. The patient may undergo treatment for cancer, e.g., radiation therapy, chemotherapy, or an immunotherapy. Later, clinicians may find it valuable to be able to perform a rapid, specific, non-invasive test for the presence of the tumor allele in the patient, as a marker of therapeutic outcome. A sample may be obtained from the patient that includes blood or plasma. The sample may include any arbitrary amount of DNA from the patient. By having previously sequenced tumor DNA and matched normal DNA from the patient, clinicians may have knowledge of a gene or other nucleic segment that may be present in a wild-type and possibly also present with a rare, tumor-specific allele. An assay may be performed that involves introducing an RNP to the sample. The RNP includes Cas endonuclease and guide RNA that hybridizes specifically to the rare, tumor-specific allele. An insight of the present disclosure is that the method may successfully detect the rare allele even when present among abundant copies of a homologous wild-type segment of DNA. Thus, where the sample includes a predominant allele and a rare allele on homologous of nucleic acid (e.g., unmutated segments of a gene from non-cancer cells with some mutated segments of the gene from cancer cells), methods of the disclosure may be used to detect the rare allele, even if present at fewer than 1% of those homologous segments. In fact, methods of the disclosure are operable to detect very rare alleles when they are present as fewer than 0.1% of copies of the gene or DNA segments. Thus certain embodiments include obtaining a sample from a patient with cancer, sequencing tumor DNA from the sample (and optionally sequencing matched normal DNA), and identifying a mutation specific to the tumor (a tumor-specific allele). Later, after the patient has undergone treatment for the cancer, a sample is obtained from the patient (preferably a blood or plasma sample which may include circulating tumor DNA (ctDNA) that includes the rare allele. The sample may include the rare allele at less than 1% of the copies of the gene in which the mutation was found, more preferably at an allele frequency of less than 0.1%. The RNP is introduced to the sample. The RNP binds to the rare allele. Unbound nucleic acid is removed, e.g., by a size-separation, a bead capture, or degradation by an exonuclease. Degrading unbound nucleic acid with exonuclease leaves the rare allele intact in the sample because the bound RNP protects the rare allele from digestion. Then the bound RNP and thus the rare allele may be detected. E.g., a sample that includes the rare allele will yield a different optical density result under spectrophotometry than a sample in which the rare allele is not present. Or the RNP can be fluorescently labelled and biotinylated then subsequently captured to a substrate (e.g., beads) and fluorescence detected.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of detecting a mutation, the method comprising:
introducing a Cas endonuclease complex to a nucleic acid sample, wherein guide RNA in the Cas endonuclease complex bind to a location of a suspected mutation;
degrading unbound nucleic acid in the sample; and
detecting presence of the mutation by detecting bound Cas endonuclease complex.

2. The method of claim 1, wherein the Cas endonuclease complex comprises a Cas endonuclease and guide RNA.

3. The method of claim 2, wherein the Cas endonuclease comprises a Cas9 protein.

4. The method of claim 3, wherein the Cas9 protein comprises a catalytically inactive Cas9 protein.

5. The method of claim 2, wherein the Cas endonuclease complex comprises a detectable label.

6. The method of claim 5, wherein the label is a fluorescent label.

7. The method of claim 5, detecting presence of the mutation by detecting presence of the label.

8. The method of claim 1, wherein degrading unbound nucleic acid comprises introducing an exonuclease to the sample.

9. The method of claim 8, wherein the method comprises deactivating the exonuclease after at least a portion of the unbound nucleic acid is digested.

10. The method of claim 9, wherein all of the unbound nucleic acid is degraded.

11. The method of claim 10, the method further comprising a wash step to isolate the mutation.

12. The method of claim 11, wherein the method comprises further analysis of the isolated mutation.

13. The method of claim 12, wherein further analysis comprises any of hybridization, spectrophotometry, sequencing, electrophoresis, amplification, fluorescence detection, and chromatography.

14. The method of claim 1, wherein the sample is a human sample.

15. The method of claim 14, wherein the sample is urine, blood, plasma, serum, sweat, saliva, semen, feces, phlegm, or a liquid biopsy.

16. The method of claim 1, wherein the sample is a non-human animal sample.

17. The method of claim 1, wherein the suspected mutation is present as a rare allele among multiple homologous segments of DNA that also include a predominant wild-type allele.

18. The method of claim 17, wherein the rare allele constitutes fewer than 0.1% of the homologous segments of DNA.

19. The method of claim 1, further comprising—prior to the introducing step—sequencing tumor DNA from a patient with cancer to identify the mutation, wherein the mutation is specific to a tumor in the patient.

20. The method of claim 19, wherein the sample comprises a blood or plasma sample from the patient and the detecting step shows the presence of tumor cells in the patient.

21. The method of claim 20, wherein the detecting step is performed months or years after treating the patient for cancer and is performed to establish success of the treatment.
